(19) United States
(12) Patent Application Publication
(10) Pub. No.: US 2017/0183813 A1
(43) Pub. Date: Jun. 29, 2017

(54) Apparatus and method for preparing hemostatic gauze

(71) Applicants: Hangzhou Singclean Medical Products Co., Ltd, Hangzhou (CN); Zhejiang University of Science and Technology, Hangzhou (CN)

(72) Inventors: Zhiguo Zhang, Hangzhou (CN); Yange Suo, Hangzhou (CN); Wei Huang, Hangzhou (CN); Weiqing Sun, Hangzhou (CN)

(21) Appl. No.: 15/201,601

(22) Filed: Jul. 4, 2016

(30) Foreign Application Priority Data
Dec. 28, 2015 (CN) 201511003048.9

(51) Int. Cl. D06M 11/64; D06M 10/02; D06M 11/38; A61L 15/28

(52) U.S. Cl. CPC D06M 11/64; A61L 15/28; D06M 10/02; D06M 11/38

(57) ABSTRACT

Disclosed are an apparatus and a method for preparing hemostatic gauze with high stability and absorptivity. The apparatus includes a tank body, a tank cover matching the tank body, stainless steel plates within the tank body, an ultrasonic generator under the tank body, a feed pipe and a liquid outlet, where the tank cover is arranged with an air inlet and an air outlet, and the feed pipe, the air inlet, the air outlet and the liquid outlet are all equipped with a valve respectively.

### CROSS-REFERENCE TO RELATED APPLICATION

This application claims priority from Chinese Patent Application No. 201511003048.9, filed on Dec. 28, 2015, which is incorporated by reference in its entirety.

### FIELD OF THE INVENTION

The present invention relates to the field of cellulose hemostatic products, in particular to an apparatus and method for preparing hemostatic gauze with high stability and absorptivity.

### BACKGROUND

With characteristics of sound biocompatibility and degradablity, the oxidized cellulose and oxidized regenerated cellulose can be used in medical field as hemostatic gauze. Currently, the medical absorbable hemostatic gauze called Surgical produced by Johnson & Johnson is the most widely used for clinical applications. With comparatively larger specific surface area, high cost and slower hemostatic speed, Surgical needs 2 to 8 minutes to stop bleeding, and is suitable for the wound with lower blood loss. But for the parts with severe blood loss, Surgical is not applicable because it is unable to absorb rapidly, timely and effectively. In addition, the existing form of the product is adverse to the combination with other products and new alternative products are in urgent need of development.

Cellulose is a natural polymeric compound formed by the attachment of D-glucopyranose. After being made into the medical gauze, the carboxyl group of the oxidized cellulose can cross-link with the calcium ions of plasma, resulting in the reaction of the oxidized cellulose and hemoglobin, which forms artificial blood clots, thereby realizing the function of hemostasis. In addition, over other hemostatic means for wounds, it has advantages of being able to degrade gradually and to be absorbed and degraded by human body, and having biocompatibility and absorbability so that it is easy to use without the necessity of being taken out.

Currently, the oxidization reactions of cellulose can be divided into selective and non-selective oxidations. In the prior art, it is intended that the oxidized cellulose is selectively oxidized, which can be divided into selective oxidation of secondary hydroxyl at positions of C2 and C3, and that of primary hydroxyl at position of C6. The oxidation systems of this sort mainly include nitrogen dioxide oxidation system and other oxidation systems of nitrogen oxides. Nevertheless, some key problems in the oxidization reactions of the cellulose by using the selective oxidation technology in the prior art remain to be solved. All the current products of the oxidized cellulose hemostatic gauze have problems, such as low intensity of gauze, insufficient storage stability, nonuniform carboxyl content, low absorptivity and uncontrollability of degradation time and the like, which restrict the application and promotion of such products.

1. The raw materials of the cellulose lack proper preprocessing means. Because the gauzes are weaved cellulose fabrics, there are a lot of slurry impurities caused during the weaving process, including moisture, inorganic and organic constituents on the surface of the raw materials of the gauze. Without proper preprocessing, these impurities would lead to lots of side reactions during the oxidation, which lower the quality of the products, and non-selective oxidation will occur, resulting in acute oxidative degradation of the macromolecular chains of the cellulose. Therefore, better preprocessing technologies must be applied to the raw materials.

2. The reaction process is not strictly controlled in the oxidation reaction system. The water content both of the raw materials and of the oxidation reaction solvent is not well controlled. The oxygen content in the reaction system also must be strictly controlled. Experiments show that in oxidation systems of nitrogen oxides, water and oxygen, when existing in the reaction system, will cause lots of side reactions including non-selective oxidations, resulting in acuter oxidative degradation of the macromolecular chains of the cellulose so that the intensity of the oxidized cellulose gauze decreases quickly.

3. During the oxidation reaction, most of the reported reaction systems are under the state of standing reaction and short of sufficient mass-transfer power, causing longer reaction time, acute degradation of the oxidized cellulose and difficulty in steadily controlling the degree of the oxidation. For example, Wang Li et al (Wang Li, Prog Pharm Sci, 2009, 33(8):365-369) adopt an HNO3/H3PO4-NaNO2 oxidation system to oxidize cellulose, where the reaction time is 96 hours and the carboxyl content of the obtained oxidized cellulose is 18.46%. 46%. CN 102018990 B provides a preparation method for oxidized cellulose hemostatic products, which uses viscose filament fabrics as starting materials and adopts an organic oxidization solvent system to oxidize the viscose filament fabrics. After the oxidation reaction, oxidized cellulose hemostatic products are prepared through washing and drying, where the reaction time is between 90 and 200 hours and the carbonyl content is between 15 and 24%.

On one hand, the concentration of the oxidation reactions of this sort is nonuniform due to the stationary state of the reaction system and the immersion of the cellulose hemostatic gauze fabrics into the same so that the oxidation reaction is also under nonuniform state. Furthermore, the by-products produced during the oxidation reaction cannot be removed timely from the fibers of the cellulose fabrics, thereby causing more side reactions, non-selective oxidation reactions especially. On the other hand, the stationary state of the reaction leads to long reaction time. However, long oxidation reaction will cause constant oxidation reaction of the reaction solvent, producing carboxylic acid substances that stick to the cellulose fabrics, which further results in quick degradation of the macromolecular chains of the cellulose as well as nonuniform distribution of the carbonyl content during the oxidation. These problems cause the performance reduction, the insufficient storage stability and the low absorptivity of the oxidized products.

4. The post-processing means for products are unreasonable. At present, it is reported in literature that after preparation by oxidation, various oxidized cellulose products are obtained only by washing for deacidification and drying treatment afterwards. However, in some reports, washing with demin-water and vacuum drying under 80° C. are adopted. According to some reports, after washing with alcohols, air drying is adopted to obtain products. Our experiments show that all these post-processing means will greatly lower the storage stability of the oxidized fiber products. As the oxidized cellulose has higher carbonyl content, its molecules are under highly active state. Therefore, to obtain hemostatic gauze with high stability and absorptivity, reasonable post-processing processes must be adopted.

To sum up, various existing methods and products have different defects, and are in urgent need of improvement.

### SUMMARY

To eliminate various defects in the prior art, the present invention provides an apparatus and method for preparing hemostatic gauze with high stability and absorptivity, which can realize integrated operation of preprocessing, oxidation reaction, washing and post-processing of the cellulose, thereby preparing hemostatic gauze with high stability and absorptivity.

For this purpose, the present invention is achieved by using the following technical solutions:

The invention discloses an apparatus for preparing hemostatic gauze with high stability and absorptivity, including a tank body, a tank cover matching the tank body, stainless steel plates within the tank body, an ultrasonic generator under the tank body, a feed pipe and a liquid outlet, where the tank cover is arranged with an air inlet and an air outlet, and the feed pipe, the air inlet, the air outlet and the liquid outlet are all equipped with a valve respectively.

As a further improvement, according to the invention, at least more than one layer of the stainless steel plate is stacked up within the tank body, and the stainless steel plates are in parallel with intervals there-between. Multi-layer stainless steel plates for placing cellulose fabrics can be stacked up within the tank body.

As a further improvement, according to the invention, the stainless steel plates are arranged with through holes and hand holes, where the through holes are circular holes evenly distributed on the stainless steel plates, and the hand holes are semicircular holes located at the middle section of both sides of the stainless steel plates. The stainless steel plates full of the circular holes can ensure sufficient contact between the reaction solution and the cellulose fabrics.

As a further improvement, according to the invention, the feed pipe includes a feeding part outside the tank body and an elbow pipe within the tank body, where the feed inlet of the feed pipe is located at the bottom of the side of the tank body, the feeding part is formed with a transparent material, and the elbow pipe contacts the bottom of the tank body and has densely distributed air holes. A reaction solution enters the tank body firstly through the feed pipe, where the feed pipe is formed with a transparent material and the liquid level within the tank body can be observed according to the theory of communicating vessels; a reactant gas fills into the tank body through the feed pipe, and flows out through the small holes of the elbow pipe at the bottom of the tank body in the form of dispersed bubbles and dissolves in the reaction solution; and NO₂ gas enters the tank body through the elbow pipe with holes at the bottom of the tank body and can rapidly dissolve in the organic solvent, reducing the waste of the NO₂ gas.

As a further improvement, according to the invention, the liquid outlet is arranged at the bottom of a side of the tank body, opposite to a feed side of the tank body. After reaction, the reaction solution is discharged from the liquid outlet and can be recycled.

As a further improvement, according to the invention, the ultrasonic generator is arranged under the tank body with a layer of the stainless steel plate between them. The ultrasonic generator under the tank body can sonicate the liquid within the tank body and strengthen the mass transfer and reaction.

The invention further discloses a preparation method for hemostatic gauze with high stability and absorptivity, including following steps:

1). Placing cellulose fabrics on the stainless steel plates;

2). Placing and stacking up the stainless steel plates layer by layer within the tank body, putting on the tank cover, where a space between the tank cover and the tank body is sealed with a PTFE gasket and the sealing is tightened with a nut;

3). Opening the valves of the feed pipe and the air outlet, connecting the air outlet to a tail gas absorber, and adding a dilute sodium hydroxide solution with the concentration of 0.5 to 5% to a specific position where all cellulose fabrics are completely immersed;

4). Sonicating with the ultrasonic generator for 5 to 2 hours to obtain an oxidized raw material of the cellulose fabrics;

5). After discharging the reaction solution through the liquid outlet, adding demin-water through the feed pipe three times to a position where the cellulose fabrics are completely immersed, and then discharging the liquid after sonicating for 10 minutes;

6). Emptying the wash water when it is neutral, and adding an anhydrous ethanol solvent until the cellulose fabrics are completely immersed, and then discharging the liquid after 10-minute ultrasonic washing;

7). Drying with N₂ gas filled in through the feed pipe and obtaining the preprocessed material of the cellulose fabrics after fully drying;

8). Introducing anhydrous cyclohexane or methyl cyclohexane through the feed pipe, where water content in the system is strictly controlled as less than 0.3%, to a position where the cellulose fabrics are completely immersed and then sonicating for 10 minutes to empty the air in the reaction system, reserving for further use;

9). Introducing NO2 gas into the reaction system, and making the concentration of the NO2 gas to be 0.2-2.1 mol/L in the oxidation reaction system;

10). Sealing the reaction system, and the oxidation reaction of the cellulose fabrics starting at room temperature;

11). With the development of the reaction, constantly produced by-products accumulating on the materials of cellulose; during the reaction, sonicating for 5 minutes every 30 minutes to accelerate the reaction and remove the by-products;

12). After 2-to-48-hour reaction, closing the valves of the feed pipe and the air outlet, and opening the liquid outlet, and introducing N₂ gas through the air inlet to discharge the reaction solution into a storage tank through the liquid outlet;

13). Opening the valves of the feed pipe and the air outlet, introducing cyclohexane or methyl cyclohexane for washing, and then discharging the liquid after sonicating for 5 minutes;

14). Using an ethanol water solution with the mass percent of 50 to 95% for immersion and washing, and discharging the liquid after sonicating for 5 minutes; when the pH of the effluent after washing is equal to 5 to 7, using anhydrous ethanol for immersion and washing again, and discharging the liquid after sonicating for 5 minutes;

15). Introducing dry N₂ gas into the reaction equipment to dry the obtained oxidized regenerated cellulose; and

16). Obtaining the completely dried products, that is, the hemostatic gauze with high stability and absorptivity, and storing and packing the hemostatic gauze in N₂ atmosphere.

As a further improvement, according to the invention, fiber fineness of the cellulose fabrics is between 0.5 and 5 deniers and fabric weight is between 40 and 500 g/m².

As a further improvement, according to the invention, reaction time is 4 to 24 hours in the step 12).

As a further improvement, according to the invention, a warranty period of the hemostatic gauze is 2 years.

The invention has following beneficial effects:

1. The invention is directed at the requirements for the preprocessing, the oxidation reaction, the washing and the post-preprocessing of the cellulose, and provides an easy-to-operate apparatus for preparing hemostatic gauze with high stability and absorptivity.

2. The raw material of the cellulose is reasonably preprocessed firstly by the apparatus. By means of the dilute sodium hydroxide solution and ultrasonic washing, lots of the slurry impurities caused during the weaving process, including moisture, inorganic and organic constituents, are removed from the surface of the raw materials of the gauze. In addition, the cellulose fabrics are immersed in advance, thereby activating the surface thereof and improving the reactivity.

3. The reaction process in the oxidation reaction system is strictly controlled. The water content of the raw material of the cellulose fabrics is strictly controlled. After immersion and washing, the raw material of the cellulose fabrics is dried to the greatest extent firstly by the anhydrous ethanol solvent through displacement and then completely dried with N₂ gas, ensuring that the system has no water, and the activated surface of the cellulose is not allowed to directly contact air. The water content of the organic solvent in the reaction system is strictly controlled further to ensure that the water content of the reaction system is less than 0.3%. After the cellulose fabrics are immersed, the small amount of gas thereof is emptied by ultrasound. Through these technology controls, the oxidation reaction is under strict control. As the system has no water and free oxygen, non-selective oxidation side reactions in the system are reduced greatly, thereby improving the performance of the products.

4. During the oxidation reaction, all the reported reaction systems are substantially under the state of standing reaction and short of sufficient mass-transfer power, causing longer reaction time, acuter degradation of the oxidized cellulose and difficulty of steadily controlling the degree of the oxidation. On one hand, the concentration of the oxidation reactions of this sort is nonuniform due to the stationary state of the reaction system and the immersion of the cellulose hemostatic gauze fabrics into the same so that the oxidation reaction is also under nonuniform state. Furthermore, the by-products produced during the oxidation reaction cannot be removed timely from the fibers of the cellulose fabrics, thereby causing more side reactions, non-selective oxidation reaction especially. On the other hand, the stationary state of the reaction leads to long reaction time. However, long oxidation reaction will cause constant oxidation reaction of the reaction solvent, producing carboxylic acid substances that stick to the cellulose fabrics, which further results in quick degradation of the macromolecular chains of the cellulose as well as nonuniform distribution of the carbonyl content during the oxidation. These problems cause the performance reduction, the insufficient storage stability and the low absorptivity of the oxidized products.

In the present application, the materials of the cellulose fabrics undergo the oxidation reaction in the sealed reaction system. With the development of the reaction, constantly produced by-products accumulate on the materials of cellulose. During the reaction, sonicating is performed for 5 minutes every 30 minutes to accelerate the reaction and remove the by-products. In this way, not too many products of the side reactions accumulate on the surface of the cellulose fabrics during the oxidation reaction and by sonicating, the by-products are removed from the surface, so the mass transfer of the reaction is accelerated and the rate of the selective oxidation reaction is increased greatly, thereby improving the performance of the products. Therefore, the reaction time in the apparatus is greatly shortened to about 2 to 48 hours, preferably 4 to 24 hours. This facilitates the rapid preparation of the products.

5. The post-processing means for the products are optimized. At present, it is reported in literature that after preparation by oxidation, various oxidized cellulose products are obtained only by washing for deacidification and drying treatment afterwards. However, in some reports, washing with demin-water and vacuum drying under 80° C. are adopted. According to some reports, after washing with alcohols, air drying is adopted to obtain products. Our experiments show that all these post-processing means will greatly lower the storage stability of the oxidized fiber products. As the oxidized cellulose has higher carbonyl content, its molecules are under highly active state. Therefore, to obtain hemostatic gauze with high stability and absorptivity, reasonable post-processing processes must be adopted.

In the present application, after the oxidation reaction, cyclohexane or methyl cyclohexane is introduced into the reaction equipment for sonicating firstly, and then ethanol water solution with the mass percent of 50 to 95% is used for immersion and sonicating. Finally, the pure oxidized cellulose hemostatic gauze is obtained after immersion and sonicating with anhydrous ethanol. At this time, the molecules of the oxidized cellulose are under highly active state, unsuitable for being directly dried or rapidly placed in air. In the present application, after washing, dry N₂ gas is introduced into the reaction equipment to dry the obtained oxidized regenerated cellulose. The completely dried products are the hemostatic gauze with high stability and absorptivity. The gauze is stored and packed in N₂ atmosphere. Therefore, the high stability of the products is ensured.

### BRIEF DESCRIPTION OF DRAWINGS

FIG. 1 is a schematic diagram of the integral structure of the preparation apparatus of the invention.

FIG. 2 is a top view of the structure of the cover for the preparation apparatus of the invention.

FIG. 3 is a top view of the structure of the stainless steel plates.

Where 1 is the feed pipe, 2 is the air inlet, 3 is the air outlet, 4 are the valves, 5 is the elbow pipe, 6 are the stainless steel plates, 7 is the liquid outlet, 8 are the circular holes, 9 are the hand holes, 10 is the ultrasonic generator, 11 is the gasket and 12 is the nut.

### DETAILED DESCRIPTION OF THE INVENTION

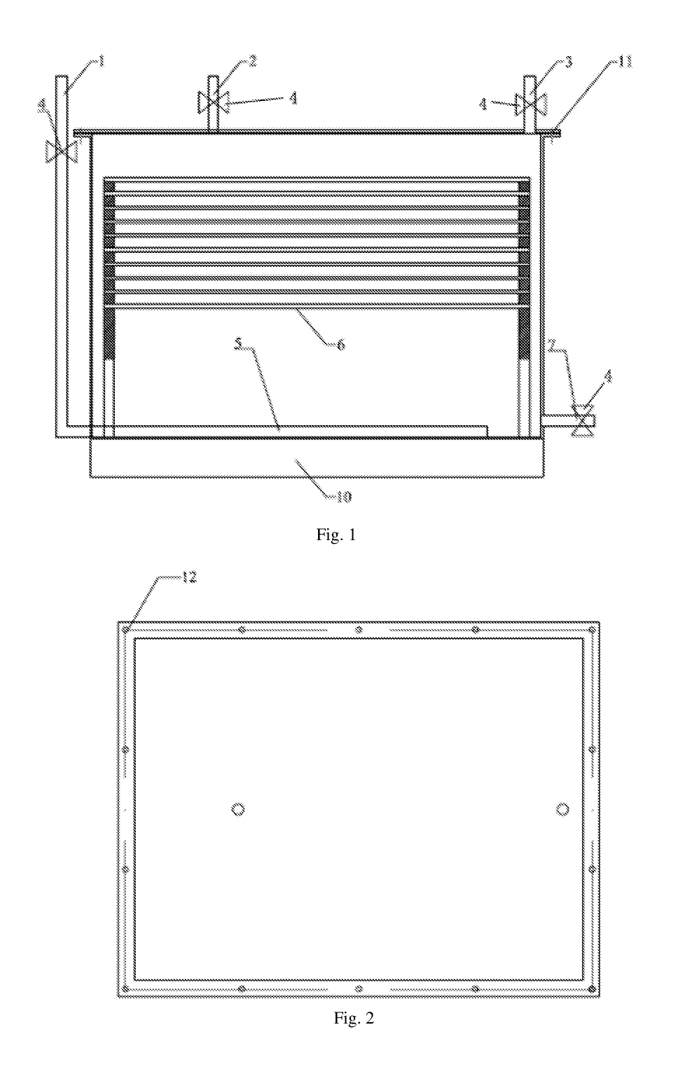

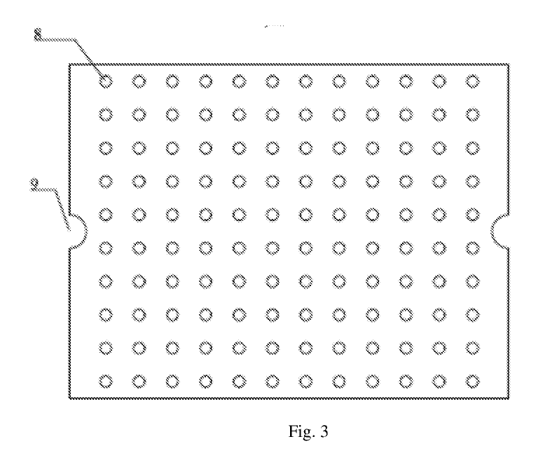

Referring to FIGS. 1-3, the present application discloses a apparatus for preparing hemostatic gauze with high stability and absorptivity, including a tank body, a tank cover matching the tank body, stainless steel plates 6 within the tank body, an ultrasonic generator 10 under the tank body, a feed pipe 1 and a liquid outlet 7, where the tank cover is arranged with an air inlet 2 and an air outlet 3, and the feed pipe 1, the air inlet 2, the air outlet 3 and the liquid outlet 7 are all equipped with a valve 4 respectively; at least more than one layer of the stainless steel plate 6 is stacked up within the tank body, and the stainless steel plates 6 are in parallel with intervals there-between each layer of steel plates, where multi-layer stainless steel plates 6 for placing cellulose fabrics can be stacked up within the tank body; the stainless steel plates are arranged with through holes and hand holes 9, where the through holes are circular holes 8 evenly distributed on the stainless steel plates 6, and the hand holes 9 are semicircular holes located at the middle section of both sides of the stainless steel plates 6, and the stainless steel plates 6 full of the circular holes 8 can ensure sufficient contact between the reaction solution and the cellulose fabrics; the feed pipe 1 includes a feeding part outside the tank body and an elbow pipe 5 within the tank body, where the feed inlet of the feed pipe 1 is located at the bottom of the side of the tank body, the feeding part is formed with a transparent material, and the elbow pipe 5 contacts the bottom of the tank body and has densely distributed air holes. A reaction solution enters the tank body firstly through the feed pipe 1, where the feed pipe 1 is formed with a transparent material and the liquid level within the tank body can be observed according to the theory of communicating vessels; a reactant gas fills into the tank body through the feed pipe 1, and flows out through the small holes of the elbow pipe 5 at the bottom of the tank body in the form of dispersed bubbles and dissolves in the reaction solution; and NO₂ gas enters the tank body through the elbow pipe 5 with holes at the bottom of the tank body and can rapidly dissolve in the organic solvent, reducing the waste of the NO₂ gas; the liquid outlet 7 is arranged at the bottom of a side of the tank body, opposite to a feed side of the tank body; after reaction, the reaction solution is discharged from the liquid outlet 7 and can be recycled; the impurities and effluent produced during the reaction will separate from the reaction solution, deposit at the bottom, and can be timely discharged through the liquid outlet 7; the ultrasonic generator 10 is arranged under the tank body with a layer of the stainless steel plate 6 between them; the ultrasonic generator 10 under the tank body can sonicate the liquid within the tank body and strengthen the mass transfer and the reaction.

The technical solutions of the invention will be further described in detail with reference to the embodiments below to illustrate the preparation method for hemostatic gauze.

### Embodiment 1

The preparation apparatus of the invention for hemostatic gauze with high stability and absorptivity is adopted, where the fiber fineness of the raw material of the cellulose fabrics is 0.5 denier, and the fabric weight is 40 g/m².

The process adopting the apparatus to prepare hemostatic gauze with high stability and absorptivity includes following steps:

Place the cellulose fabrics with a weight of 400 g on the stainless steel plates 6.

Place and stack up the stainless steel plates 6 layer by layer within the tank body, put on the tank cover, where the space between the tank cover and the tank body is sealed with the PTFE gasket 11 and the sealing is tightened with the nut 12.

Open the valves 4 of the feed pipe 1 and the air outlet 3, and connect the air outlet 3 to the tail gas absorber. Add the dilute sodium hydroxide solution with the concentration of 0.5% to a specific position where all cellulose fabrics are completely immersed.

Sonicate for 2 hours to obtain the oxidized raw material of the cellulose fabrics.

After discharging the reaction solution, add demin-water three times to a position where the cellulose fabrics are completely immersed, and then discharge the liquid after sonicating for 10 minutes.

Empty the wash water when it is neutral and add the anhydrous ethanol solvent until the cellulose fabrics are completely immersed, and then discharge the liquid after sonicating for 10 minutes.

Dry with N₂ gas, and obtain the preprocessed material of the cellulose fabrics after fully drying.

Introduce the anhydrous cyclohexane through the feed pipe 1, where the water content in the system is strictly controlled as less than 0.3%, to a position where the cellulose fabrics are completely immersed, and then sonicate for 10 minutes to empty the air in the reaction system. Reserve for further use.

Introduce NO₂ gas into the reaction system, and make the concentration of the NO₂ gas to be 0.2 mol/L in the oxidation reaction system.

Seal the reaction system, and the oxidation reaction of the cellulose fabrics starts at room temperature

With the development of the reaction, constantly produced by-products accumulate on the materials of the cellulose fabrics. During the reaction, sonicate for 5 minutes every 30 minutes to accelerate the reaction and remove the by-products.

After 24-hour reaction, close the valves 4 of the feed pipe 1 and the air outlet 3, open the liquid outlet 7, and introduce N₂ gas through the air inlet 2 to discharge the reaction solution into the storage tank.

Open the valves 4 of the feed pipe 1 and the air outlet 3, introduce cyclohexane into the reaction equipment for washing, and then discharge the liquid after sonicating for 5 minutes.

Use the ethanol water solution with the mass percent of 50% for immersion and washing, and discharging the liquid after sonicating for 5 minutes. When the pH of the effluent after washing is equal to 5, use anhydrous ethanol for immersion and washing again, and discharge the liquid after sonicating for 5 minutes.

Introduce the dry N₂ gas into the reaction equipment to dry the obtained oxidized regenerated cellulose.

The completely dried products are the hemostatic gauze with high stability and absorptivity. The gauze is stored and packed in N₂ atmosphere.

The carbonyl content of the products is 18. 5%, and the warranty period is 2 years.

### Embodiment 2

The preparation apparatus of the invention for hemostatic gauze with high stability and absorptivity is adopted, where the fiber fineness of the raw material of the cellulose fabrics is 2 deniers, and the fabric weight is 200 g/m².

The process adopting the apparatus to prepare hemostatic gauze with high stability and absorptivity includes following steps

Place the cellulose fabrics with a weight of 1000 g on the reaction apparatus.

Place and stack up the stainless steel plates 6 layer by layer within the tank body, and put on the tank cover, where the space between the tank cover and the tank body is sealed with the PTFE gasket 11 and the sealing is tightened with the nut 12.

Open the valves 4 of the feed pipe 1 and the air outlet 3, connect the air outlet 3 to the tail gas absorber, and add the dilute sodium hydroxide solution with the concentration of 1% to a specific position where all cellulose fabrics are completely immersed.

Sonicate for 1 hour to obtain the oxidized raw material of the cellulose fabrics.

After discharging the reaction solution, add demin-water three times to a position where the cellulose fabrics are completely immersed, and then discharge the liquid after sonicating for 10 minutes.

Empty the wash water when it is neutral and add the anhydrous ethanol solvent until the cellulose fabrics are completely immersed, and then discharge the liquid after sonicating for 10 minutes.

Dry with N₂ gas, and obtain the preprocessed material of the cellulose fabrics after fully drying.

Introduce the methyl cyclohexane through the feed pipe 1, where the water content in the system is strictly controlled as less than 0.3%, to a position where the cellulose fabrics are completely immersed, and then sonicate for 10 minutes to empty the air in the reaction system. Reserve for further use.

Introduce NO₂ gas into the reaction system, and make the concentration of the NO₂ gas to be 0.5 mol/L in the oxidation reaction system.

Seal the reaction system, and the oxidation reaction of the cellulose fabrics starts at room temperature

With the development of the reaction, constantly produced by-products accumulate on the material of the cellulose fabrics. During the reaction, sonicate for 5 minutes every 30 minutes to accelerate the reaction and remove the by-products.

After 12-hour reaction, close the valves 4 of the feed pipe 1 and the air outlet 3, open the liquid outlet 7, and introduce N₂ gas through the air inlet 2 to discharge the reaction solution into the storage tank.

Open the valves 4 of the feed pipe 1 and the air outlet 3, introduce methyl cyclohexane into the reaction equipment, and then discharge the liquid after sonicating for 5 minutes.

Use the ethanol water solution with the mass percent of 75% for immersion and washing, and discharge the liquid after sonicating for 5 minutes. When the pH of the effluent after washing is equal to 6, use anhydrous ethanol for immersion and washing again, and discharge the liquid after sonicating for 5 minutes.

Introduce the dry N₂ gas into the reaction equipment to dry the obtained oxidized regenerated cellulose.

The completely dried products are the hemostatic gauze with high stability and absorptivity. The gauze is stored and packed in N₂ atmosphere.

The carbonyl content of the products is 23. 5%, and the warranty period is 2 years.

### Embodiment 3

The preparation apparatus of the invention for hemostatic gauze with high stability and absorptivity is adopted, where the fiber fineness of the raw material of the cellulose fabrics is 5 deniers, and the fabric weight is 500 g/m².

The process adopting the apparatus to prepare hemostatic gauze with high stability and absorptivity includes following steps:

Place the cellulose fabrics with a weight of 1000 g on the reaction equipment.

Place and stack up the stainless steel plates 6 layer by layer within the tank body, and put on the tank cover, where the space between the tank cover and the tank body is sealed with the PTFE gasket 11 and the sealing is tightened with the nut 12.

Open the valves 4 of the feed pipe 1 and the air outlet 3, connect the air outlet 3 to the tail gas absorber, and add the dilute sodium hydroxide solution with the concentration of 5% to a specific position where all cellulose fabrics are completely immersed.

Sonicate for 0.5 hour to obtain the oxidized raw material of the cellulose fabrics.

After discharging the reaction solution, add demin-water three times to a position where the cellulose fabrics are completely immersed, and then discharge the liquid after sonicating for 10 minutes.

Empty the wash water when it is neutral, and add the anhydrous ethanol solvent until the cellulose fabrics are completely immersed, and then discharge the liquid after sonicating for 10 minutes.

Dry with N₂ gas, and obtain the preprocessed material of the cellulose fabrics after fully drying.

Introduce the anhydrous cyclohexane through the feed pipe 1, where the water content in the system is strictly controlled as less than 0.3%, to a position where the cellulose fabrics are completely immersed, and then sonicate for 10 minutes to empty the air in the reaction system. Reserve for further use.

Introduce NO₂ gas into the reaction system, and make the concentration of the NO₂ gas to be 2.1 mol/L in the oxidation reaction system.

Seal the reaction system, and the oxidation reaction of the cellulose fabrics starts at room temperature.

With the development of the reaction, constantly produced by-products accumulate on the materials of the cellulose fabrics. During the reaction, sonicate for 5 minutes every 30 minutes to accelerate the reaction and remove the by-products.

After 4-hour reaction, close the valves 4 of the feed pipe 1 and the air outlet 3, and open the liquid outlet 7, and introduce N₂ gas through the air inlet 2 to discharge the reaction solution into the storage tank.

Open the valves 4 of the feed pipe 1 and the air outlet 3, introduce cyclohexane into the reaction equipment for washing, and then discharge the liquid after sonicating for 5 minutes.

Use the ethanol water solution with the mass percent of 95% for immersion and washing, and discharge the liquid after sonicating for 5 minutes. When the pH of the effluent after washing is equal to 7, use anhydrous ethanol for immersion and washing again, and discharging the liquid after sonicating for 5 minutes.

Introduce the dry N₂ gas into the reaction equipment to dry the obtained oxidized regenerated cellulose.

The completely dried products are the hemostatic gauze with high stability and absorptivity. The gauze is stored and packed in N₂ atmosphere.

The carbonyl content of the products is 20.0%, and the warranty period is 2 years.

In the end, it is to be noted that what is illustrated is only the embodiments of the invention. Apparently, the invention is not limited to the embodiments but can include more variations. Any variation directly inferred from or associated with the disclosures of the invention by those of ordinary skill in the art should be considered within the protection scope of the invention.

What is claimed is:

1. An apparatus for preparing hemostatic gauze, wherein the apparatus comprises a tank body, a tank cover matching the tank body, stainless steel plates (6) within the tank body, an ultrasonic generator (10) under the tank body, a feed pipe (1) and a liquid outlet (7), wherein the tank cover is arranged with an air inlet (2) and an air outlet (3), and the feed pipe (1), the air inlet (2), the air outlet (3) and the liquid outlet (7) are all equipped with a valve (4) respectively.

2. The apparatus according to claim 1, wherein at least more than one layer of the stainless steel plate (6) is stacked up within the tank body, and the stainless steel plates (6) are parallel with intervals there-between.

3. The apparatus according to claim 2, wherein the stainless steel plates are arranged with through holes and hand holes (9), wherein the through holes are circular holes (8) evenly distributed on the stainless steel plates (6), and the hand holes (9) are semicircular holes located at the middle section of both sides of the stainless steel plates (6).

4. The apparatus according to claims 1, wherein the feed pipe (1) comprises a feeding part outside the tank body and an elbow pipe (5) within the tank body, wherein the feed inlet of the feed pipe (1) is located at the bottom of the side of the tank body, the feeding part is formed with a transparent material, and the elbow pipe (5) contacts the bottom of the tank body and has densely distributed air holes.

5. The apparatus according to claim 4, wherein the liquid outlet (7) is arranged at the bottom of a side of the tank body, opposite to a feed side of the tank body, and the liquid outlet (7) is at a distance of 1 cm or more from the bottom of the tank body.

6. The apparatus according to claim 4, wherein the ultrasonic generator (10) is arranged under the tank body with a layer of the stainless steel plate (6) between them.

7. The apparatus according to claim 2, wherein the feed pipe (1) comprises a feeding part outside the tank body and an elbow pipe (5) within the tank body, wherein the feed inlet of the feed pipe (1) is located at the bottom of the side of the tank body, the feeding part is formed with a transparent material, and the elbow pipe (5) contacts the bottom of the tank body and has densely distributed air holes.

8. The apparatus according to claim 3, wherein the feed pipe (1) comprises a feeding part outside the tank body and an elbow pipe (5) within the tank body, wherein the feed inlet of the feed pipe (1) is located at the bottom of the side of the tank body, the feeding part is formed with a transparent material, and the elbow pipe (5) contacts the bottom of the tank body and has densely distributed air holes.

9. A method for preparing hemostatic gauze using the apparatus according to claim 1, comprising the following steps:
1) placing cellulose fabrics on the stainless steel plates (6);
2) placing and stacking up the stainless steel plates (6) layer by layer within the tank body, putting on the tank cover, wherein a space between the tank cover and the tank body is sealed with a PTFE gasket and the sealing is tightened with a nut;
3) opening the valves (4) of the feed pipe (1) and the air outlet (3), connecting the air outlet (3) to a tail gas absorber, adding a dilute sodium hydroxide solution with the concentration of 0.5 to 5% to a specific position where all cellulose fabrics are completely immersed;
4) sonicating with the ultrasonic generator (10) for 0.5 to 2 hours to obtain an oxidized raw material of the cellulose fabrics;
5) after discharging the reaction solution through the liquid outlet (7), adding demin-water through the feed pipe (1) three times to a position where the cellulose fabrics are completely immersed, and then discharging the liquid after sonicating for 10 minutes;
6) emptying the wash water when it is neutral, and adding an anhydrous ethanol solvent until the cellulose fabrics are completely immersed, and then discharging the liquid after sonicating for 10 minutes;
7) drying with N₂ gas filled in through the feed pipe (1) and obtaining the preprocessed material of the cellulose fabrics after fully drying;
8) introducing anhydrous cyclohexane or methyl cyclohexane through the feed pipe (1), wherein water content in the system is strictly controlled as less than 0.3%, to a position where the cellulose fabrics are completely immersed, and then sonicating for 10 minutes to empty the air in the reaction system, reserving for further use;
9) introducing NO₂ gas into the reaction system, and making the concentration of the NO₂ gas to be 0.2 to 2.1 mol/L in the oxidation reaction system;
10) sealing the reaction system, and the oxidation reaction of the cellulose fabrics starting at room temperature;
11) with the development of the reaction, constantly produced by-products accumulating on the materials of the cellulose fabrics; during the reaction, sonicating for 5 minutes every 30 minutes to accelerate the reaction and remove the by-products;
12) after 2-to-48-hour reaction, closing the valves (4) of the feed pipe (1) and the air outlet (3), and opening the liquid outlet (7), and introducing N₂ gas through the air inlet (2) to discharge the reaction solution into a storage tank through the liquid outlet (7);
13) opening the valves (4) of the feed pipe (1) and the air outlet (3), introducing cyclohexane or methyl cyclohexane for washing, and then discharging the liquid after sonicating for 5 minutes;
14) using an ethanol water solution with the mass percent of 50 to 95% for immersion and washing, and discharging the liquid after sonicating for 5 minutes; when the pH of the effluent after washing is equal to 5 to 7, using absolute ethyl alcohol for immersion and washing again, and discharging the liquid after sonicating for 5 minutes;
15) introducing dry N₂ gas into the reaction equipment to dry the obtained oxidized regenerated cellulose; and
16) obtaining the completely dried products, that is, the hemostatic gauze with high stability and absorptivity, and storing and packing the hemostatic gauze in N₂ atmosphere.

10. The method according to claim 9, wherein fiber fineness of the cellulose fabrics is between 0.5 and 5 deniers and fabric weight is between 40 and 500 g/m².

11. The method according to claim 9, wherein reaction time of the step 12) is 4 to 24 hours.

12. The method according to claim 9, wherein a warranty period of the hemostatic gauze is 2 years.

13. A method for preparing hemostatic gauze using the apparatus according to claim 2, comprising the following steps:
1) placing cellulose fabrics on the stainless steel plates (6);
2) placing and stacking up the stainless steel plates (6) layer by layer within the tank body, putting on the tank cover, wherein a space between the tank cover and the tank body is sealed with a PTFE gasket and the sealing is tightened with a nut;
3) opening the valves (4) of the feed pipe (1) and the air outlet (3), connecting the air outlet (3) to a tail gas absorber, adding a dilute sodium hydroxide solution with the concentration of 0.5 to 5% to a specific position where all cellulose fabrics are completely immersed;
4) sonicating with the ultrasonic generator (10) for 0.5 to 2 hours to obtain an oxidized raw material of the cellulose fabrics;
5) after discharging the reaction solution through the liquid outlet (7), adding demin-water through the feed pipe (1) three times to a position where the cellulose fabrics are completely immersed, and then discharging the liquid after sonicating for 10 minutes;
6) emptying the wash water when it is neutral, and adding an anhydrous ethanol solvent until the cellulose fabrics are completely immersed, and then discharging the liquid after sonicating for 10 minutes;
7) drying with N₂ gas filled in through the feed pipe (1) and obtaining the preprocessed material of the cellulose fabrics after fully drying;
8) introducing anhydrous cyclohexane or methyl cyclohexane through the feed pipe (1), wherein water content in the system is strictly controlled as less than 0.3%, to a position where the cellulose fabrics are completely immersed, and then sonicating for 10 minutes to empty the air in the reaction system, reserving for further use;
9) introducing NO₂ gas into the reaction system, and making the concentration of the NO₂ gas to be 0.2 to 2.1 mol/L in the oxidation reaction system;
10) sealing the reaction system, and the oxidation reaction of the cellulose fabrics starting at room temperature;
11) with the development of the reaction, constantly produced by-products accumulating on the materials of the cellulose fabrics; during the reaction, sonicating for 5 minutes every 30 minutes to accelerate the reaction and remove the by-products;
12) after 2-to-48-hour reaction, closing the valves (4) of the feed pipe (1) and the air outlet (3), and opening the liquid outlet (7), and introducing N₂ gas through the air inlet (2) to discharge the reaction solution into a storage tank through the liquid outlet (7);
13) opening the valves (4) of the feed pipe (1) and the air outlet (3), introducing cyclohexane or methyl cyclohexane for washing, and then discharging the liquid after sonicating for 5 minutes;
14) using an ethanol water solution with the mass percent of 50 to 95% for immersion and washing, and discharging the liquid after sonicating for 5 minutes; when the pH of the effluent after washing is equal to 5 to 7, using absolute ethyl alcohol for immersion and washing again, and discharging the liquid after sonicating for 5 minutes;
15) introducing dry N₂ gas into the reaction equipment to dry the obtained oxidized regenerated cellulose; and
16) obtaining the completely dried products, that is, the hemostatic gauze with high stability and absorptivity, and storing and packing the hemostatic gauze in N₂ atmosphere.

14. A method for preparing hemostatic gauze using the apparatus according to claim 3, comprising the following steps:
1) placing cellulose fabrics on the stainless steel plates (6);
2) placing and stacking up the stainless steel plates (6) layer by layer within the tank body, putting on the tank cover, wherein a space between the tank cover and the tank body is sealed with a PTFE gasket and the sealing is tightened with a nut;
3) opening the valves (4) of the feed pipe (1) and the air outlet (3), connecting the air outlet (3) to a tail gas absorber, adding a dilute sodium hydroxide solution with the concentration of 0.5 to 5% to a specific position where all cellulose fabrics are completely immersed;
4) sonicating with the ultrasonic generator (10) for 0.5 to 2 hours to obtain an oxidized raw material of the cellulose fabrics;
5) after discharging the reaction solution through the liquid outlet (7), adding demin-water through the feed pipe (1) three times to a position where the cellulose fabrics are completely immersed, and then discharging the liquid after sonicating for 10 minutes;
6) emptying the wash water when it is neutral, and adding an anhydrous ethanol solvent until the cellulose fabrics are completely immersed, and then discharging the liquid after sonicating for 10 minutes;
7) drying with N₂ gas filled in through the feed pipe (1) and obtaining the preprocessed material of the cellulose fabrics after fully drying;
8) introducing anhydrous cyclohexane or methyl cyclohexane through the feed pipe (1), wherein water content in the system is strictly controlled as less than 0.3%, to a position where the cellulose fabrics are completely immersed, and then sonicating for 10 minutes to empty the air in the reaction system, reserving for further use;
9) introducing NO₂ gas into the reaction system, and making the concentration of the NO₂ gas to be 0.2 to 2.1 mol/L in the oxidation reaction system;
10) sealing the reaction system, and the oxidation reaction of the cellulose fabrics starting at room temperature;
11) with the development of the reaction, constantly produced by-products accumulating on the materials of the cellulose fabrics; during the reaction, sonicating for 5 minutes every 30 minutes to accelerate the reaction and remove the by-products;
12) after 2-to-48-hour reaction, closing the valves (4) of the feed pipe (1) and the air outlet (3), and opening the liquid outlet (7), and introducing N₂ gas through the air inlet (2) to discharge the reaction solution into a storage tank through the liquid outlet (7);
13) opening the valves (4) of the feed pipe (1) and the air outlet (3), introducing cyclohexane or methyl cyclohexane for washing, and then discharging the liquid after sonicating for 5 minutes;
14) using an ethanol water solution with the mass percent of 50 to 95% for immersion and washing, and discharging the liquid after sonicating for 5 minutes; when the pH of the effluent after washing is equal to 5 to 7, using absolute ethyl alcohol for immersion and washing again, and discharging the liquid after sonicating for 5 minutes;
15) introducing dry N₂ gas into the reaction equipment to dry the obtained oxidized regenerated cellulose; and
16) obtaining the completely dried products, that is, the hemostatic gauze with high stability and absorptivity, and storing and packing the hemostatic gauze in N₂ atmosphere.

15. A method for preparing hemostatic gauze using the apparatus according to claim 5, comprising the following steps:
1) placing cellulose fabrics on the stainless steel plates (6);
2) placing and stacking up the stainless steel plates (6) layer by layer within the tank body, putting on the tank cover, wherein a space between the tank cover and the tank body is sealed with a PTFE gasket and the sealing is tightened with a nut;
3) opening the valves (4) of the feed pipe (1) and the air outlet (3), connecting the air outlet (3) to a tail gas absorber, adding a dilute sodium hydroxide solution with the concentration of 0.5 to 5% to a specific position where all cellulose fabrics are completely immersed;
4) sonicating with the ultrasonic generator (10) for 0.5 to 2 hours to obtain an oxidized raw material of the cellulose fabrics;
5) after discharging the reaction solution through the liquid outlet (7), adding demin-water through the feed pipe (1) three times to a position where the cellulose fabrics are completely immersed, and then discharging the liquid after sonicating for 10 minutes;
6) emptying the wash water when it is neutral, and adding an anhydrous ethanol solvent until the cellulose fabrics are completely immersed, and then discharging the liquid after sonicating for 10 minutes;
7) drying with N₂ gas filled in through the feed pipe (1) and obtaining the preprocessed material of the cellulose fabrics after fully drying;
8) introducing anhydrous cyclohexane or methyl cyclohexane through the feed pipe (1), wherein water content in the system is strictly controlled as less than 0.3%, to a position where the cellulose fabrics are completely immersed, and then sonicating for 10 minutes to empty the air in the reaction system, reserving for further use;
9) introducing NO₂ gas into the reaction system, and making the concentration of the NO₂ gas to be 0.2 to 2.1 mol/L in the oxidation reaction system;
10) sealing the reaction system, and the oxidation reaction of the cellulose fabrics starting at room temperature;
11) with the development of the reaction, constantly produced by-products accumulating on the materials of the cellulose fabrics; during the reaction, sonicating for 5 minutes every 30 minutes to accelerate the reaction and remove the by-products;
12) after 2-to-48-hour reaction, closing the valves (4) of the feed pipe (1) and the air outlet (3), and opening the liquid outlet (7), and introducing N₂ gas through the air inlet (2) to discharge the reaction solution into a storage tank through the liquid outlet (7);
13) opening the valves (4) of the feed pipe (1) and the air outlet (3), introducing cyclohexane or methyl cyclohexane for washing, and then discharging the liquid after sonicating for 5 minutes;
14) using an ethanol water solution with the mass percent of 50 to 95% for immersion and washing, and discharging the liquid after sonicating for 5 minutes; when the pH of the effluent after washing is equal to 5 to 7, using absolute ethyl alcohol for immersion and washing again, and discharging the liquid after sonicating for 5 minutes;
15) introducing dry N₂ gas into the reaction equipment to dry the obtained oxidized regenerated cellulose; and
16) obtaining the completely dried products, that is, the hemostatic gauze with high stability and absorptivity, and storing and packing the hemostatic gauze in N₂ atmosphere.

16. A method for preparing hemostatic gauze using the apparatus according to claim 6, comprising the following steps:
1) placing cellulose fabrics on the stainless steel plates (6);
2) placing and stacking up the stainless steel plates (6) layer by layer within the tank body, putting on the tank cover, wherein a space between the tank cover and the tank body is sealed with a PTFE gasket and the sealing is tightened with a nut;
3) opening the valves (4) of the feed pipe (1) and the air outlet (3), connecting the air outlet (3) to a tail gas absorber, adding a dilute sodium hydroxide solution with the concentration of 0.5 to 5% to a specific position where all cellulose fabrics are completely immersed;
4) sonicating with the ultrasonic generator (10) for 0.5 to 2 hours to obtain an oxidized raw material of the cellulose fabrics;
5) after discharging the reaction solution through the liquid outlet (7), adding demin-water through the feed pipe (1) three times to a position where the cellulose fabrics are completely immersed, and then discharging the liquid after sonicating for 10 minutes;
6) emptying the wash water when it is neutral, and adding an anhydrous ethanol solvent until the cellulose fabrics are completely immersed, and then discharging the liquid after sonicating for 10 minutes;
7) drying with N₂ gas filled in through the feed pipe (1) and obtaining the preprocessed material of the cellulose fabrics after fully drying;
8) introducing anhydrous cyclohexane or methyl cyclohexane through the feed pipe (1), wherein water content in the system is strictly controlled as less than 0.3%, to a position where the cellulose fabrics are completely immersed, and then sonicating for 10 minutes to empty the air in the reaction system, reserving for further use;
9) introducing NO₂ gas into the reaction system, and making the concentration of the NO₂ gas to be 0.2 to 2.1 mol/L in the oxidation reaction system;
10) sealing the reaction system, and the oxidation reaction of the cellulose fabrics starting at room temperature;
11) with the development of the reaction, constantly produced by-products accumulating on the materials of the cellulose fabrics; during the reaction, sonicating for 5 minutes every 30 minutes to accelerate the reaction and remove the by-products;
12) after 2-to-48-hour reaction, closing the valves (4) of the feed pipe (1) and the air outlet (3), and opening the liquid outlet (7), and introducing N₂ gas through the air inlet (2) to discharge the reaction solution into a storage tank through the liquid outlet (7);
13) opening the valves (4) of the feed pipe (1) and the air outlet (3), introducing cyclohexane or methyl cyclohexane for washing, and then discharging the liquid after sonicating for 5 minutes;
14) using an ethanol water solution with the mass percent of 50 to 95% for immersion and washing, and discharging the liquid after sonicating for 5 minutes; when the pH of the effluent after washing is equal to 5 to 7, using absolute ethyl alcohol for immersion and washing again, and discharging the liquid after sonicating for 5 minutes;
15) introducing dry N₂ gas into the reaction equipment to dry the obtained oxidized regenerated cellulose; and
16) obtaining the completely dried products, that is, the hemostatic gauze with high stability and absorptivity, and storing and packing the hemostatic gauze in N₂ atmosphere.
